(19) United States
(12) Patent Application Publication
(10) Pub. No.: US 2020/0181608 A1
(43) Pub. Date: Jun. 11, 2020

(54) METHOD FOR KNOCKING OUT TARGET GENE IN T CELL IN VITRO AND crRNA USED IN THE METHOD

(71) Applicants: Jiangsu Hengrui Medicine Co., Ltd., Lianyungang, Jiangsu (CN); Shanghai Hengrui Pharmaceutical Co., Ltd., Minhang District, Shanghai (CN)

(72) Inventors: Zuohan PENG, Minhang District, Shanghai (CN); Lianjun SHEN, Minhang District, Shanghai (CN); Weikang TAO, Minhang District, Shanghai (CN)

(21) Appl. No.: 16/623,605

(22) PCT Filed: Jun. 19, 2018
(86) PCT No.: PCT/CN2018/091804

§ 371 Date: Dec. 17, 2019

(30) Foreign Application Priority Data
Jun. 20, 2017 (CN) 201710469539.5

(51) Int. Cl. C12N 15/11; C12N 9/22; C12N 15/85; C12N 5/0783; A61K 35/17

(52) U.S. Cl. CPC C12N 15/11; C12N 9/22; C12N 15/85; C12N 2510/00; A61K 35/17; C12N 2800/80; C12N 2310/20; C12N 5/0636

(57) ABSTRACT

Provided is a method for knocking out target genes in T cells in vitro based on the CRISPR-Cas9 system. Also provided are crRNAs that target the TRAC, B2M and PD1 genes, and a kit comprising sgRNA formed by linking the crRNA and tracrRNA corresponding to a Cas9 protein, the Cas9 protein, an oligo deoxyribonucleic acid (N-oligo) or a milt DNA fragment. The kit is used to knock out the TCR, B2M and/or PD1 genes in T cells. Also provided are T cells with gene knockout, obtained according to a method of the present invention and uses thereof.

### FIELD OF THE INVENTION

The invention belongs to the field of biological medicine. Specifically, it relates to a method for knocking out target genes in T cells in vitro, a crRNA used in the method, the T cells obtained by the method and use thereof.

### BACKGROUND OF THE INVENTION

Adoptive cell therapy (ACT), involving the transfer of autologous antigen-specific T cells produced ex vivo, is a promising strategy for the treatment of viral infections and cancers. T cells used in adoptive cell therapy may be generated by amplification of antigen-specific T cells or by redirection of genetically designed T cells (Park, Rosenberg et al. Trends Biotechnol. 2011, 29(11): 550-557).

CAR-T is obtained by genetically inserting a chimeric antigen receptor (CAR) into isolated T cell to enhance the targeting, killing activity and persistence of the T cell, and the recognition of tumor cell surface antigens is independent of the MHC restriction. CARs consist of an extracellular antigen binding region, a transmembrane region, and an intracellular signal transduction region of T cell receptor (such as CD3ζ and costimulatory molecules). The extracellular antigen binding region is composed of a light chain variable region (VL) linked to a heavy chain variable region (VH) of monoclonal antibody via hinge, to form a single chain fragment variable (scFv), which is capable of recognizing a specific tumor antigen. Relevant clinical trials have shown that CAR has a better therapeutic effect in patients with lymphoma which is not responsive to other treatments. The study on CART-19 by Carl June of the University of Pennsylvania showed that 45 out of 75 patients with leukemia (including adults and children) had complete remission after treatment with CAR-T cells.

However, the existing CAR-T therapies typically utilize autologous lymphocytes from tumor patients. Such lymphocytes are modified, cultured, activated, and expanded in vitro and then returned back to the patient. In addition to side effects such as cytokine storm, there are three major problems: firstly, CAR-T treatment is not applicable to advanced patients with a lower number or poorer quality of lymphocytes; secondly, the efficacy of CAR-T therapy in solid tumors is still not significant, probably due to the poorer survival rate and the lower activity of immune cells in tumor tissues resulting from the influence of the signal pathway of the immunosuppression checkpoint; finally, the cost is high and the burden on the patient is increased, because CAR-T is an personalized treatment. Therefore, the development of universal CAR-T cells (UCART) derived from allogeneic origin will promote its application.

In the early stage, gene knock-out was performed by a targeting vector via homologous recombination or by RNAi technology, but both have problems, such as cumbersome operation and low efficiency. Cellectis has successfully cured several children patients suffering from relapsed acute lymphocytic leukemia (ALL) by directionally knocking out the T-cell receptor alpha (TCRα) gene (reducing graft versus host disease) and CD52 gene (making cells tolerant to Alemtuzumab) with the allogeneic CAR-T therapy, UCART19, developed by TALEN technology. However, cumbersome construction and large-scale sequencing processes are needed when knocking out TCR through TALEN as proposed by Cellectis. Currently, clustered regularly interspaced short palindromic repeat (CRISPR-associated, CRISPR-Cas9) achieves gene editing by recognizing specific DNA sequences, and this is simpler and more efficient than TALEN.

Currently, there are examples of gene knockout based on CRISPR/Cas9 system, such as those mentioned in CN104395463A, CN105518146A and CN106191062A. However, there are still some problems to be solved, such as low knockout rate of the target gene, requiring several transfection or transformation processes, cumbersome operations, much damage to the T cells, or high off-target rate. Therefore, there is still a need to optimize the method for knocking out the TCR gene based on CRISPR/Cas9 system and also crRNAs with higher accuracy.

### SUMMARY OF THE INVENTION

The object of the present invention is to overcome the problems present in immunotherapy in the prior art, and to provide a method for constructing TCR-negative T cells by knocking out TCR with CRISPR/Cas9 gene editing technology, and to provide TCR-negative T cells obtained by the method.

One object of the present invention is to provide TCR and PD1 (or B2M) double negative T cells and to provide a method for constructing the same.

Another object of the present invention is to provide TCR, B2M and PD1 triple negative T cells and to provide a method for constructing the same.

Further, the above TCR-negative T cells, TCR and PD-1 (or B2M⁻) double negative T cells and TCR/B2M/PD1 triple negative T cells are sorted by using magnetic beads, and are used in adoptive cell immunotherapy of tumors and the like.

In a first embodiment, a method for knocking out one or more target gene(s) in T cells in vitro is provided, comprising the steps of:

1) contacting sgRNAs targeting one or more target gene(s) in the T cells with Cas9 protein respectively, to form a protein-RNA complex (RNP);

2) mixing said RNP with an oligo deoxyribonucleic acid (N-oligo) or a milt DNA fragment, and transforming the obtained mixture into T cells, wherein the sgRNAs direct the Cas9 protein to the corresponding target sequences of the target gene and to hybridize with the target sequences, thereby the target gene is cleaved, and the cleavage efficiency of the target gene is not less than 75%.

In a preferred embodiment, in a method for knocking out one or more target gene(s) in T cells in vitro of the invention, the target gene is one or more selected from the group consisting of TRAC, TRBC, B2M and PD1 genes, and the sgRNA targets a coding sequence of the target gene or a regulatory sequence for the expression of the target gene.

Further, in a method for knocking out one or more target gene(s) in T cells in vitro of the invention, the sgRNA consists of, from 5′ to 3′: a crRNA targeting the target gene and having a length of 17-20 nucleotides (nt), which is linked to a tracrRNA corresponding to the Cas9 protein, wherein the crRNA is preferably 17 nt in length.

In a preferred embodiment, in a method for knocking out one or more target gene(s) in T cells in vitro of the invention, the oligo deoxyribonucleic acid is a double-stranded DNA with a length of 100-250 base pairs (bp) or a single-stranded DNA with a length of 100 -250 nt.

In a preferred embodiment, in a method for knocking out one or more target gene(s) in T cells in vitro of the invention, the crRNA targeting the TRAC gene is any one or more selected from the group consisting of the crRNAs shown in SEQ ID NOs: 1-12, the sequence of the crRNA targeting the B2M gene is shown in SEQ ID NO: 13, and the crRNA targeting the PD1 gene is any one or more selected from the group consisting of the crRNAs shown in SEQ ID NOs: 14-16.

In a preferred embodiment, in a method for knocking out one or more target gene(s) in T cells in vitro of the invention, the Cas9 protein is a Cas9 protein derived from Streptococcus pyogenes, and the sequence thereof is shown in SEQ ID NO: 18.

In a preferred embodiment, in a method for knocking out one or more target gene(s) in T cells in vitro of the invention, the Cas9 protein is a Cas9 protein derived from Streptococcus pyogenes, and the sequence of the tracrRNA corresponding to the Cas9 protein is shown in SEQ ID NO: 17.

In a preferred embodiment, in a method for knocking out one or more target gene(s) in T cells in vitro of the invention, the T cell is selected from the group consisting of helper T cells, cytotoxic T cells, memory T cells, regulatory T cells, natural killer T cells, γδT cells, CAR-T cells, and TCR-T cells.

In another aspect, the present invention further provides T cells with target gene(s) knocked out, wherein the T cells are obtained by the method described above.

In another aspect, the invention further provides a crRNA for use in knocking out a target gene, wherein the crRNA comprises one or more sequences selected from the group consisting of SEQ ID NOs: 1-16.

In a preferred embodiment, provided is the crRNA used for knocking out a target gene out in the present invention targets a coding sequence of the target gene or targets a regulatory sequence for the expression of the target gene, wherein the target gene is one or more selected from the group consisting of TRAC, TRBC, B2M and PD1 genes.

In a preferred embodiment, provided is the crRNAs for use in knocking out a target gene in the present invention, wherein the target gene is the TRAC gene, and the crRNA is one or more selected from the group consisting of those shown in SEQ ID NOs: 1-12.

In a preferred embodiment, provided is the crRNAs for use in knocking out a target gene in the present invention, wherein the target gene is the B2M gene, and the sequence of the crRNA is set forth in SEQ ID NO: 13.

In a preferred embodiment, provided is the crRNAs for use in knocking out a target gene in the present invention, wherein the target gene is the PD1 gene, and the crRNA is one or more selected from the group consisting of those shown in SEQ ID NOs: 14-16.

In another aspect, the present invention also provides a sgRNA for use in knocking out a target gene, the sgRNA consists of a crRNA linked to a tracrRNA corresponding to Cas9 protein, and wherein the crRNA comprises one or more sequences selected from the group consisting of SEQ ID NOs: 1-16.

In a preferred embodiment, provided are the sgRNAs for use in knocking out a target gene according to the present invention, wherein the target gene is one or more selected from the group consisting of TRAC, TRBC, B2M and PD1 genes.

In a preferred embodiment, provided are the sgRNAs for use in knocking out the target gene TRAC according to the present invention, wherein the sgRNA consists of a crRNA linked to a tracrRNA corresponding to Cas9 protein, and the crRNA is one or more selected from the group consisting of those shown in SEQ ID NOs: 1-12.

In a preferred embodiment, provided are the sgRNAs for use in knocking out target gene B2M according to the present invention, wherein the sgRNA consists of a crRNA linked to a tracrRNA corresponding to Cas9 protein, and the crRNA is shown in SEQ ID NO: 13.

In a preferred embodiment, provided are the sgRNAs for use in knocking out target gene PD-1 according to the present invention, wherein the sgRNA consists of a crRNA linked to a tracrRNA corresponding to Cas9 protein, and the crRNA is one or more selected from the group consisting of those shown in SEQ ID NOs: 14-16.

In a preferred embodiment, provided are the sgRNAs for use in knocking out target gene according to the present invention, wherein the Cas9 protein is a Cas9 protein derived from Streptococcus pyogenes, with amino acid of SEQ ID NO: 18.

In a preferred embodiment, provided is the sgRNAs for use in knocking out target gene according to the present invention, wherein the sequence of the tracrRNA corresponding to the Cas9 protein is shown in SEQ ID NO: 17.

In another aspect, the invention provides a gene knockout kit, wherein the kit comprises:

a: one or more crRNA(s) as described above, or one or more sgRNA(s) as described above;

b: Cas9 protein;

c: an oligo deoxyribonucleic acid or a milt DNA fragment.

In a preferred embodiment, in the kit used for knocking out genes, the oligo deoxyribonucleic acid is a double-stranded DNA with a length of 100-250 bp or a single-stranded DNA with a length of 100-250 nt.

In a preferred embodiment, in the kit used for knocking out genes, the Cas9 protein is a Cas9 protein derived from Streptococcus pyogenes, and the sequence of the tracrRNA corresponding to the Cas9 protein is shown in SEQ ID NO: 17.

In some embodiments, the invention provides the use of T cells involving gene been knockout of the invention for the preparation of anti-tumor drugs.

In some embodiments, the present invention also provides use of the T cells involving gene been knockout of the invention for the preparation of a medicament for preventing or treating infectious diseases caused by viruses or bacteria.

In some embodiments, TCR, B2M or PD1 are effectively knocked out by using the designed crRNA and the method. The killing activity in vitro of CAR-T cells with knocking out of TCR and B2M and/or PD1 gene(s), is not affected due to the knockout of TCR, B2M and/or PD1 genes.

### DESCRIPTION OF THE DRAWINGS

FIG. 1: Comparison of knockout efficiencies of different delivery systems. The results show that the RNP delivery mode has the highest efficiency in knocking out genes in Jurkat cells.

FIGS. 2A-2B: Effect of N-oligo on efficiency of CRISPR-Cas9-based system in knocking out genes in T cells. FIG. 2A shows a comparison of efficiency in knocking out genes in T cells; FIG. 2B shows a comparison of efficiency in knocking out genes in CAR-T cells.

FIG. 3: Effect of milt DNA fragments on efficiency in gene knockout in T cells.

FIG. 4: Detection of efficiency in B2M gene knockout.

FIG. 5: Detection of the effect of the screened crRNAs on PD1 gene knockout.

FIGS. 6A-6B: Analysis of gene mutations caused by RNP and N-Oligo or milt DNA. FIG. 6A shows analytic results for TRAC, and FIG. 6B shows analytic results for B2M.

FIGS. 7A-7C: Analysis of RNP off-target rate. FIG. 7A shows analytic results of the off-target rate of the TRAC gene; FIG. 7B shows analytic results of the off-target rate of the B2M gene; and FIG. 7C shows analytic results of the off-target rate of the PD1 gene.

FIGS. 8A-8B: Analysis of activation of CD25 and CD69 in T cells with TRAC gene knocked out. FIG. 8A shows a comparison of CD69 activation; and FIG. 8B shows a comparison of CD25 activation.

### DETAILED DESCRIPTION OF THE INVENTION

In one aspect, the invention provides a method for altering a target gene in a cell.

The study described herein demonstrates the use of the allele targeting approach of CRISPR/Cas system to produce mutant cells with an efficiency up to 80%. In particular, the work described herein surprisingly and unexpectedly demonstrates a multiplex guide strategy that provides a method for specifically identifying useful RNA guide sequences, as well as specific guide sequences suitable for targeting specific genes (e.g., TRAC, TRBC, B2M, PD1).

CRISPR systems are used for the methods and compositions provided by the present invention, such systems also include those described in International Publication No. WO 2013142578 A1 and WO 2013098244 A1, which are incorporated herein entirely by reference.

An exemplary method for altering polynucleotide sequences of a target gene in a cell comprises contacting the polynucleotide sequences with a CRISPR (Clustered Regularly Interspaced Short Palindromic Repeats) sequence-related protein (Cas) and one or two ribonucleic acids, thereby a RNP is formed, wherein the ribonucleic acids direct the Cas protein to a target motif of the polynucleotide sequence of the target gene and to hybridize with the target motif, wherein the polynucleotide sequence of the target gene is cleaved, and the alternation efficiency of cells transformed with the RNP is 75% or more.

Any means for altering the polynucleotide sequence of a target gene is contemplated in the present invention, said means are readily available to the skilled artisan by using the CRISPR/Cas system of the present invention. Any CRISPR/Cas system capable of altering the polynucleotide sequence of a target gene in a cell can be used. Such CRISPR/Cas systems can employ a variety of Cas proteins (Haft et al. PLoS Comput Biol. 2005; 1(6) e60). Such Cas proteins allow the CRISPR/Cas system to alter the polynucleotide sequence of the target gene in the cell, including RNA binding proteins, endonucleases and exonucleases, helicases, and polymerases. In some embodiments, the CRISPR/Cas system is a CRISPR Type I system. In some embodiments, the CRISPR/Cas system is a CRISPR Type II system.

The CRISPR/Cas system of the present invention can be used to alter the polynucleotide sequence(s) of a target gene in a cell. The present invention contemplates altering the polynucleotide sequence(s) of a target gene in a cell for any purpose. In some embodiments, the polynucleotide sequence(s) of a target gene in a cell is/are altered to produce a mutant cell.

An exemplary Cas9 protein, herein, may be a Streptococcus pyogenes Cas9 protein or a functional portion thereof In some embodiments, the Cas9 protein is a Cas9 protein derived from any bacterial species or a functional portion thereof. The Cas9 protein is a member of the Type II CRISPR system, which typically includes a trans-encoded small RNA (tracrRNA), an endogenous ribonuclease 3 (rnc), and a Cas9 protein.

CrRNA (CRISPR-derived RNA) and tracrRNA (trans-activating RNA) are genetically engineered and are ligated together to obtain sgRNA (single guide RNA). Finally, a complex is formed by the sgRNA-expressing sequence and Cas9 is transformed into a cell, thereby the target genes can be knocked out.

In some embodiments, such alteration corrects some undesired polynucleotide sequences of the target gene into desired sequences. The CRISPR/Cas system of the invention can be used to correct any type of mutation or error in the polynucleotide sequence of a target gene. For example, the CRISPR/Cas system of the invention can be used to insert the nucleotide sequences which have been missed from the polynucleotide sequences of a target gene due to deletion. In some examples, the CRISPR/Cas system of the present invention can also be used to delete or excise some nucleotide sequences caused by an insertion mutation from the polynucleotide sequences of a target gene. In some embodiments, the CRISPR/Cas system of the invention can be used to replace incorrect nucleotide sequences with correct nucleotide sequences (e.g., to recover the impaired function due to the functional mutation of polynucleotide sequences of a target gene, i.e. SNP).

The CRISPR/Cas system of the present invention can unexpectedly cleave target genes with high efficiency, when compared with conventional CRISPR/Cas systems. In certain embodiments, the efficiency of cleaving of the target gene is at least about 5%. In certain embodiments, the efficiency of cleaving of the target gene is at least about 10%. In certain embodiments, the efficiency of cleaving of the target gene is from about 10% to about 80%. In certain embodiments, the efficiency of cleaving of the target gene is from about 30% to about 80%. In certain embodiments, the efficiency of cleaving of the target gene is from about 50% to about 80%. In some embodiments, the efficiency of cleaving of the target gene is greater than or equal to about 75%, or greater than or equal to about 80%.

In some embodiments, the target gene is a genome. In some embodiments, the target gene is a human genome. In some embodiments, the target gene is a mammalian genome. In some embodiments, the target gene is a vertebrate genome.

The CRISPR/Cas system of the present invention can be applied in various applications to knock out the polynucleotide sequences or a portion thereof in the target gene. For example, knocking out the target gene polynucleotide sequence in a cell can be performed in vitro for research purposes. For ex vivo purpose, knocking out the target gene polynucleotide sequence(s) in a cell is applicable for treating or preventing disorders associated with the expression of the target gene polynucleotide sequence(s) (e.g., knocking out the mutant allele(s) in the cells ex vivo; and introducing those cells with mutant allele(s) that have been knocked out back into the subject).

In another aspect, the invention provides a method for treating or preventing disorders associated with the expression of the polynucleotide sequences in a subject.

In order to understand the present invention easily, certain technical and scientific terms are specifically defined below. All of the other technical and scientific terms used herein have the meaning commonly understood by one of ordinary skill in the art to which this invention belongs, unless otherwise explicitly defined herein.

### I. Terminology

As used herein, the term “contacting” (i.e., contacting a polynucleotide sequence with a clustered regularly interspaced short palindromic repeats-associated (Cas) protein and/or ribonucleic acid) is intended to include incubating the Cas protein and/or ribonucleic acids in the cell together in vitro (e.g., adding the Cas protein or nucleic acid encoding the Cas protein to cells in culture) or contacting a cell ex vivo. The step of contacting the target gene polynucleotide sequence with a Cas protein and/or ribonucleic acid as disclosed herein can be conducted in any suitable manner. For example, the cells can be treated in adherent or suspension culture. It is understood that cells contacted with Cas protein and/or ribonucleic acid as disclosed herein may also be simultaneously or subsequently contacted with another agent, such as a growth factor or other differentiation agent or environment, to stabilize or to differentiate the cells further.

The term “treating” and the like, as applied to an isolated cell, include subjecting the cell to any kind of process or condition, or performing any kind of manipulation or procedure on the cell. As applied to a subject, the term refers to providing a cell in which a target gene polynucleotide sequence has been altered ex vivo according to the method described herein to an individual. The individual is typically suffering from diseases or injury, or is at increased risk of suffering from diseases relative to an average member of the population and in need of such attention, care or management.

As used herein, the term “treating” refers to administering to a subject an effective amount of cells with target polynucleotide sequences altered ex vivo according to the method described herein, so that the subject has a reduction in at least one symptom of the disease or an improvement in the disease, for example, beneficial or desired clinical results. For the purpose of the present invention, beneficial or desired clinical results include, but are not limited to, alleviation of one or more symptoms, diminishment of extent of the disease, stabilized state (i.e., not deterioration) of the disease, delay or slowing the disease progression, amelioration or palliation of the disease state, and remission (whether partial or total), whether detectable or undetectable. Treatment may refer to prolonging survival, as compared to the expected survival without treatment. Thus, one of skill in the art recognizes that a treatment may improve a disease condition, but may not be a complete cure for the disease. As used herein, the term “treatment” includes prophylaxis. Alternatively, treatment is considered to be “effective” if the progression of a disease is reduced or halted. “Treatment” can also refer to prolonging survival as compared to expected survival without treatment. Those in need of treatment include those already diagnosed with a disorder associated with expression of the polynucleotide sequence, as well as those likely to develop such a disorder due to genetic susceptibility or other factors.

As used herein, a “mutant cell” refers to a cell with a resulting genotype that differs from the original genotype. In some examples, “a mutant cell” exhibits a mutant phenotype, for example, when a normally functioning gene is altered using the CRISPR/Cas system of the invention. In other examples, “a mutant cell” exhibits a wild-type phenotype, for example, when a mutated genotype is corrected by using the CRISPR/Cas system of the invention. In some embodiments, the target polynucleotide sequence(s) in a cell is/are altered to correct or repair the genetic mutation (e.g., to restore the normal genotype of the cell). In some embodiments, the target gene polynucleotide sequence(s) in a cell is/are altered to induce a genetic mutation (e.g., to destroy the function of a gene or genome element).

In some embodiments, the alteration is an indel. “Indel” as used herein refers to a mutation resulting from an insertion, deletion or combination thereof. As will be appreciated by those skilled in the art, an indel in the coding region of a genomic sequence will result in a frameshift mutation, unless the length of the indel is a multiple of three. In some embodiments, the alteration is a point mutation. “Point mutation” as used herein refers to the substitution of one of the nucleotides. The CRISPR/Cas system of the invention can be used to induce an indel of any length or point mutation in a target polynucleotide sequence.

“Oligo deoxyribonucleic acid” or “N-oligo” refers to a deoxyribonucleic acid fragment with a random sequence which has been transformed into a cell together with RNP, when the RNP delivery system is used to knock out a gene. Preferably, it is a double-stranded DNA with 100-250 bp in length, or a single-stranded DNA with 100-250 nt in length.

“Milt DNA fragment” refers to a small molecule fragment obtained by cutting milt DNA by mechanically shearing a solution containing salmon sperm DNA. For example, a 1% salmon sperm DNA solution is repeatedly beaten with a 7-gauge needle to cut the DNA into small molecules, aliquoted and stored.

“Knockout” as used herein includes deleting all or a portion of the target polynucleotide in a manner that interferes with the function of the target polynucleotide sequence. For example, a knockout can be achieved by altering a target gene polynucleotide sequence by inducing an indel in the functional domain (e.g., in the DNA binding domain) of the target polynucleotide sequence. Based on the details described herein, those skilled in the art will readily appreciate how to use the CRISPR/Cas systems of the present invention to knock out the target polynucleotide or a portion thereof.

In some embodiments, cleavage of the target gene results in decreased expression of the target gene. The term “decreased” is generally used herein to mean a statistically significant decreased amount. However, for avoidance of doubt, “decreased” means a decrease by at least 10% as compared to a reference level, for example decreased by at least about 20%, or at least about 30%, or at least about 40%, or at least about 50%, or at least about 60%, or at least about 70%, or at least about 75%, or at least about 80%, or at least about 90%, or up to and including a 100% decrease (i.e., absent level as compared to the a reference sample) , or any decrease between 10% and 100% as compared to a reference level.

The term “statistically significant” or “significantly” refers to statistical significance and generally means a two standard deviation (2SD) below normal, or lower, concentration of the marker. The term refers to statistical evidence for the presence of a difference. It is defined as a probability of making a decision to reject the null hypothesis when the null hypothesis is actually true. The decision is often made using the p-value.

In some embodiments, cleavage of the target gene is cleavage of a homozygous target gene. In some embodiments, cleavage of the target gene is cleavage of a hybrid target gene.

Cas9 protein (also known as CRISPR-related endonuclease Cas9/Csn1) is a polypeptide comprising 1368 amino acids. An exemplary amino acid sequence of a Cas9 protein is shown in SEQ ID NO: 18. Cas9 contains two endonuclease domains, including the RuvC-like domain (residues 7-22, 759-766, and 982-989), which cleaves target DNA that is noncomplementary to crRNA; and an HNH nuclease domain (residues 810-872), which cleaves target DNA that is complementary to crRNA.

T cell receptor (TCR) is a hetero-dimeric protein receptor that presents a specific antigenic peptide on the Major Histocompatibility Complex (MHC). In the immune system, the binding of antigen-specific TCR to the peptide-loaded MHC (pMHC) complex triggers direct physical contact between T cells and Antigen Presenting Cells (APCs), and then the other cell membrane surface molecules on T cells interact with those on APCs. This leads to a series of subsequent cell signaling and other physiological responses, allowing different antigen-specific T cells to exert an immune effect on their target cells.

TCR is a glycoprotein present on the cell membrane surface in the form of heterodimer of alpha chain/beta chain or gamma chain/delta chain. The TCR heterodimer in 95% of T cells consists of alpha and beta chains, while 5% of T cells have a TCR consisting of gamma and delta chains. The native αβ heterodimeric TCR has alpha chain and beta chain, and the alpha chain and the beta chain constitute the subunit of the αβ heterodimeric TCR. Broadly, each of the alpha and beta chains comprises a variable region, a junction region, and a constant region, and the beta chain typically also contains a short diversity region between the variable region and the junction region, but the diversity region is often considered as a part of the junction region. Each variable region comprises three CDRs (complementarity determining regions), CDR1, CDR2 and CDR3, which are interspersed among the framework regions. The CDR regions determine the binding of the TCR to the pMHC complex, wherein the CDR3 is recombinantly formed by the variable region and the junction region and is referred to as the hypervariable region. The alpha and beta chains of TCR are generally considered to have two “domains”, namely a variable domain and a constant domain, and the variable domain consists of the variable region linked to the junction region. The constant domain sequence of the TCR can be found in the public database of the International Immunogenetics Information System (IMGT). For example, the constant domain sequence of α chain of the TCR molecule is “TRAC*01”, and the constant domain sequence of β chain of the TCR molecule is “TRBC1*01” or “TRBC2*01”. In addition, the alpha and beta chains of TCR also contain transmembrane and cytoplasmic regions, wherein the cytoplasmic region is very short.

B2M, also known as beta-2 microglobulin, is the light chain of MHC class I molecules and is therefore an indispensable part of MHC. In humans, B2M is encoded by the b2m gene located on chromosome 15, and opposite to other MHC genes as a cluster of genes located on chromosome 6. The protein derived from human consists of 119 amino acids and has a molecular weight of 11,800 Daltons. A murine model with β-2 microglobulin deficiency has demonstrated that B2M is necessary for expressing MHC class I on cell surface and for the stability of peptide-binding cleft.

“PD-1” or “PD1” is a type I transmembrane receptor with a molecular weight of 50-55 kDa, which was originally identified in T cells suffering from activation-induced apoptosis. PD-1 is expressed on T cells, B cells and macrophages. The ligands of PD-1 are members of the B7 family, PD-L1 (B7-H1) and PD-L2 (B7-DC).

PD-1 is a member of the immunoglobulin (Ig) superfamily and contains a single IgV-like domain in its extracellular region. The PD-1 cytoplasmic domain contains two tyrosines, the one that is closer to the membrane (VAYEEL in mouse PD-1) is located within ITIM (the inhibitory motif of the immunoreceptor tyrosine). The ITIM on PD-1 predicts that this molecule acts by recruiting cytosolic phosphatase to attenuate the signaling of antigen receptors. The human and murine PD-1 proteins share approximately 60% amino acid identity, with four potentially conserved N-glycosylation sites and residues defining the Ig-V domain. The cytoplasmic ITIM and the ITIM-like motif around the carboxy terminal tyrosine (TEYATI in human and mice) are also conserved between human and murine orthologues.

### II. Examples and Test Examples

The invention is further described in the following examples, but these examples are not intended to limit the scope of the invention.

Experimental methods, for which the specific conditions are not specifically indicated in the examples or test examples of the present invention, were generally carried out according to conventional conditions or according to the conditions recommended by the manufacturers. Reagents, for which the sources are not specifically indicated, were routinely purchased from the market.

### EXAMPLES

### Example 1: PBMC Extraction

Healthy volunteers with no cold and fever symptoms were recruited, and informed consents were signed. 100 ml of blood was taken from the vein into a BD anticoagulant tube by medical professionals. The blood was mixed with an equal amount of PBS buffer (containing 2% fetal bovine serum). 15 ml of Ficoll buffer (GE healthcare) was added into a PBMC separation tube Sepmate-50 (STEMCELL Technology), and the mixture of blood and PBS was then added. The pellet was resuspended in PBS after centrifugation. The resuspended cells were counted, 10 μl of the suspension was added with 10 μl of 0.1% trypan blue, mixed; cell numbers and survival rates were counted.

### Example 2: Purification of T Cells

PBMCs were centrifuged at 300 g for 5 minutes, the supernatant was discarded, and added with corresponding amount of PBS buffer (containing 2 mM EDTA and 1% fetal bovine serum), the cells were resuspended and the cell density was adjusted to 5×10⁷/ml. Human T cells were purified by using EasySep™ Human T Cell Enrichment Kit available from STEMCELL Technology. First, 50 μl/ml of Cooktail was added to the PBMC suspension, mixed well and placed at room temperature for 10 minutes. Then, 50 μl/ml of EasySep™ D Magnetite Particles were added, mixed well, and placed at room temperature for 5 minutes. The cell suspension was added to a 5 ml flow tube and placed in magnetic poles for 5 minutes. The cell suspension was quickly decanted, PBS buffer was added into the flow tube and resuspended, repeated 3 times. The obtained cell suspension was centrifuged at 300 g for 5 minutes, the supernatant was discarded, and the cell pellets were resuspended in VIVO-15 medium available from LONZA. The density was adjusted to 1×10⁶/ml, and rIL-2 (R&D) was added to make the concentration 100 IU/ml. Then, the cells were cultured in a 37° C. cell culture incubator.

### Example 3: T Cell Activation

Anti-CD3/anti-CD28 magnetic beads (Life Technology) were resuspended in PBS buffer (containing 2 mM EDTA and 1% fetal bovine serum), then placed in magnetic poles for 2 minutes, and then the supernatant was discarded. The above process was repeated 4 times. The magnetic beads were washed, and then added into the purified T cells in a ratio of 1:1, mixed and cultured at 37° C. for 3 days. After 3 days, the magnetic beads were removed, and the target cells were resuspended several times by pipetting. The cell suspension was placed in magnetic poles, placed for two minutes, and the magnetic beads on the tube wall were discarded.

### Example 4: Infecting T Cells with CAR Virus

Construction of CAR Lentiviral Plasmid:

The structure of CD19 CAR from the outside to the inside is CD19 scFv, Hinge structure, transmembrane structure, 4-1BB and CD3z. An expression vector was constructed with CD19 CAR and vector pHR-CAR. The lentiviral plasmid pHR-CAR and the two helper plasmids dR8.91 and pCMV-VSV-G were extracted by using Plasmid Extraction maxi Kit available from Tiangen.

Package and Concentration of CAR Lentivirus:

293T cells (purchased from ATCC) were overgrown on a 75 cm² culture dish one day before transfection, and passaged at 1:3 with 15 ml of culture medium for each dish. Transfection was carried out according to the procedure of Lipo3000. The transfection system was as follows:

| Transfection system 1 | Transfection system 2 |
|---|---|
| pHR-CAR: 7.5 μg | |
| dR8.91: 5.625 μg | |
| pCMV-VSV-G: 1.875 μg | |
| Opti-MEM (Gibco): 700 μl | Opti-MEM (Gibco): 700 μl |
| P3000: 30 μl | Lipofectamine: 36 μl |

The system 1 was mixed well with the system 2, incubated for 5 minutes and mixed again, and then incubated for another 10 minutes. 293T cells were carefully added. 6 hours later, the medium was replaced with fresh medium. 48 hours later, the culture medium was collected and stored at 4° C. 15 ml of fresh medium was added again, and the supernatant was collected 24 hours later. The obtained virus supernatant was filtered through 0.45 μm filter and added in ultracentrifuge tube. The tubes were centrifuged at 50000 g for 2 hours and 45 minutes at 4° C., the supernatant was carefully and thoroughly removed, and the white virus pellet visible to naked eye was resuspended with PBS buffer at a volume of 1% of the supernatant volume. The resuspended virus was dissolved at 4° C. for about 30 minutes. After completely dissolved, it was aliquoted and stored in a refrigerator at −80° C.

Infecting T Cells with CAR Lentivirus:

The human primary T cells were activated with anti-CD3/anti-CD28 magnetic beads. One day later, the cells were resuspended and placed in magnetic poles for two minutes. The cell suspension was then taken and cell counting was performed. About 1×10⁷ cells were centrifuged at 300 g for 5 minutes, the medium was discarded, and 1 ml of fresh medium was added to resuspend the cells. Concentrated lentivirus was added to adjust the MOI to 5, and mixed well. The tubes were centrifuged at 2000 g for 90 minutes at 32° C., the supernatant was discarded, and fresh medium (100 IU/ml rIL-2) was added to adjust the cell density to 1×10⁶ cells/ml. The cells were resuspended and combined with the newly isolated anti-CD3/anti-CD28 magnetic beads. The cultivation was continued in an incubator at 37° C. CAR-T cells were obtained.

### Example 5: Knockout of TCR, B2M, PD1 Gene

(1) Design of crRNA

An appropriate target region was selected based on the nucleotide sequences of TRAC, B2M and PD1, and a crRNA with length of 17-20 nt was designed. A sgRNA was formed by ligating the crRNA with a tracrRNA corresponding to Cas9 protein used herein. The crRNAs with high knockout efficiency and low off-target rates were screened in experiments. The sequences of the selected crRNAs were as follows:

TABLE 1
crRNAs against target genes

| Target | crRNA | | |
|---|---|---|---|
| gene | name | sequence | SEQ ID NO. |
| TRAC | crRNA-1 | CAAAUGUGUCACAAAGUA | 1 |
| | crRNA-2 | AAAACUGUGCUAGACAUG | 2 |
| | crRNA-3 | UCAAGAGCAACAGUGCUG | 3 |
| | crRNA-4 | CACCUUCUUCCCCAGCCC | 4 |
| | crRNA-5 | GAAUAAUGCUGUUGUUGA | 5 |
| | crRNA-6 | GAUUUAGAGUCUCUCAGC | 6 |
| | crRNA-7 | ACGGCAGGGUCAGGGUUC | 7 |
| | crRNA-8 | GUUCCUGUGAUGUCAAGC | 8 |
| | crRNA-9 | UCAAAACCUGUCAGUGAU | 9 |
| | crRNA-10 | GAAUCCUCCUCCUGAAAG | 10 |
| | crRNA-11 | GGUACACGGCAGGGUCA | 11 |
| | crRNA-12 | GAGAAUCAAAAUCGGUGAAU | 12 |
| B2M | crRNA-13 | GUAGCGCGAGCACAGCUA | 13 |
| PD1 | crRNA-14 | CGACUGGCCAGGGCGCCUGU | 14 |
| | crRNA-15 | GUGCUACAACUGGGCUGG | 15 |
| | crRNA-16 | GGCGCCCUGGCCAGUCGUCU | 16 |

Cas9 protein is derived from Streptococcus Pyogenes (Cas9 Nuclease NLS, S. Pyogenes (BioLabs)), the corresponding tracrRNA sequence (SEQ ID NO: 17) was:

GUUUUAGAGCUAGAAAUAGCAAGUUAAAUAAGGCUAGUCCGUUAUCAA
CUUGAAAAAGUGGCACCGAGUCGGUGCUUUU

The amino acid sequence of the Cas9 (including NLS) protein used herein (SEQ ID NO: 18):

MDKKYSIGLAIGTNSVGWAVITEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAKNLSDAILL
SDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIRE
VKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYS
LFELENGRKRMLASAGELQKGNELALPSKYVNFLYLASHYEKLKGSPEDN
EQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSI
TGLYETRIDLSQLGGDPKKKRKVMDK

The sgRNA consisting of the crRNA shown in Table 1 linked to tracrRNA corresponding to Cas9 protein described above was prepared, wherein the crRNA was located at the 5′ end of the tracrRNA.

(2) In Vitro Transcription of sgRNA:

PCR amplification of sgRNA template was performed first:

| Reaction System (20 μl) | | PCR condition | |
|---|---|---|---|
| plasmid | 1 μl (0.5 pg) | 98° C. | 30 sec |
| 5 × HF buffer | 4 μl | 98° C. | 10 sec |
| dNTP (10 mM) | 0.4 μl | 60° C. | 25 sec |
| primer-F(10 μm) | 0.4 μl | 72° C. | 2 min |
| primer-R(10 μm) | 0.4 μl | 72° C. | 10 min |
| Phusion | 0.2 μl | 38 times | |
| H₂O | 13.6 μl | 4° C. | 10 min |

Then, PCR products were recovered:

The Manual for Regular DNA Product Purification Kit DP214 was available from Tiangen and was used as a reference. DNA for in vitro sgRNA transcription was obtained. The sgRNA was transcribed with in vitro transcription kit MEGAshortscript™ Kit (Cat # AM1354) available from Ambion. The manual for Ambion MEGAclear™ Kit cat # AM1908 was used as a reference. The obtained sgRNAs were purified and detected by spectrophotometer and denatured agarose gel electrophoresis, all of them were qualified and aliquoted immediately for use.

(3) TRAC Gene was Knocked Out in T Cells Via Electroporation of CRISPR-Cas9:

The obtained CAR-T cells were electro-transformed (this method is also applied to knocking out primary T cells), mainly using the LONZA 4D electroporation instrument, and the kit used was P3 Primary Cell 4D-Nucleofector™ X kit.

First, the electroporation system was prepared: 10 μl of Nucleofector buffer, 30 μg of Cas9 protein (about 9 μg/μl) and 4 μg of sgRNA were mixed and incubated at room temperature for 10 minutes. CAR-T cells were activated for three days, and then the anti-CD3/anti-CD28 magnetic beads were removed via magnetic poles. 5×10⁶ cells/tube were taken and centrifuged at 300 g for 5 minutes to completely remove the supernatant. The incubated electroporation system was added to the cell pellet, further 72 μl of Nucleofector buffer and 18 μl of Supplement buffer were added, mixed well and added to 100 μl of LONZA electroporation cuvette, which was placed in LONZA-4D electroporation instrument and electroporation was carried out according to the E0-115 procedure. After the electroporation was completed, the electroporation cuvette was allowed to stand at room temperature for 5 minutes. The cells were transferred from the electroporation cuvette into the pre-warmed VIVO-15 medium, cell density was adjusted to 1×10⁶ cells/ml, and cultured at 37° C.

### Example 6: Screening of TCR-Negative Cells

After TRAC was knocked out from CAR-T cells with CRISPR-Cas9, the CAR-T cells were cultured until day 10, and TCR-negative cells were enriched. First, all of cells were centrifuged at 300 g for 5 minutes, washed twice with PBS buffer (containing 2 mM EDTA and 1% fetal bovine serum). The cell density was adjusted to 1×10⁷ cells/ml, and then combined with 100 μl/ml of Biotin-TCR antibody (purchased from Miltenyi Biotec, Germany), and incubated for 10 minutes at 4° C. in the darkness. After washing once with PBS buffer, the cell density was adjusted to 1×10⁷ cells/ml. Anti-Biotin Microbeads were added at 50 μl/ml, and kept at 4° C. for 15 minutes in the darkness. After washing once with PBS buffer, the cells were resuspended in 500 μl of buffer. LD column (purchased from Miltenyi Biotec) was placed in magnetic poles and rinsed with 2 ml of PBS buffer once. Then, 500 μl of the cell suspension was added, the target cells flowed through the LD column and were collected from the bottom of the LD column. When all of the cell suspension flowed out, 2 ml of PBS buffer was loaded onto the LD column, repeated twice. The collected cell suspension was centrifuged at 300 g for 5 minutes, and resuspended in pre-warmed medium.

### TEST EXAMPLES

### Test Example 1: The Most Optimized crRNA was Chosen for CRISPR-Cas9-Based Knockout of TRAC

The crRNA sequences designed for TRAC as indicated in Example 5 were compared in the test. sgRNA was obtained after in vitro transcription, and then Cas9 protein was electroporated into the activated primary T cells. 48 hours later, the extracellular expression of the TCR protein was detected by flow cytometer. The results showed that all of the tested crRNAs can knock out the TRAC gene to varying degrees. Among them, crRNA-11 exhibited the highest knockout efficiency.

### Test Example 2: Comparative Analysis of Different Delivery Systems

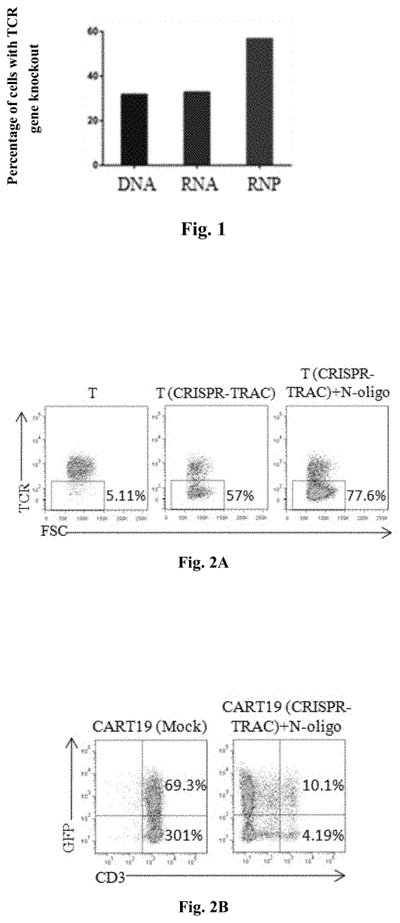

Three delivery systems tested were plasmid, mRNA and RNP (protein-RNA complex), respectively; crRNA was designed to target TRAC, and maxi plasmid extraction was performed according to Example 4. For in vitro transcription of Cas9 mRNA, a DNA template containing T7 promoter was firstly obtained by PCR with T7 primers; then, the Cas9 mRNA was obtained by in vitro transcription with T7 in vitro Transcription Kit available from Ambion. sgRNA and Cas9 protein complex were obtained in the same way as those indicated in Example 5.5×10⁶ Jurkat cells were centrifuged and the supernatant was discarded. Electroporation was performed with three different delivery materials respectively on Electroporation System Neon MPK5000 (Invitrogen). 48 hours later, 0.5×10⁶ cells were taken and washed twice with PBS buffer, resuspended with 100 μl of buffer, and then added with 10 μl of PE-TCR antibody (eBioscience), mixed well and incubated at 4° C. for 30 minutes. After washing once using PBS buffer, the cells were resuspended with 500 μl of buffer and loaded onto the TCR detection channel of the flow cytometer. The results are shown in FIG. 1.

### Test Example 3: Random N-Oligo or Milt DNA Increases the Efficiency of CRISPR-Cas9 in Knocking Out TRAC

When the RNP delivery system was used to knock out a gene, RNP was mixed well with random sequences of N-oligo (oligo deoxyribonucleic acid) or milt DNA and simultaneously transformed by electroporation.

An exemplary sequence of N-oligo:

(SEQ ID NO: 19)
TCATGTGGTCGGGGTAGCGGCTGAAGCACTGCACGCCGTACGTCAGGGT
GGTCACGAGGGTGGGCCAGGGCACGGGCAGCTTGCCGGTGGTGCAGATG
AACTTCAGGGTCAGCTTGCCGTAGGTGGC

On the basis of the disclosure of Example 5 section (3), 100-200 nM of N-oligo DNA was additionally added to the RNP complex, wherein the N-oligo DNA was Page-grade. The effect of N-oligo on the efficiency of CRISPR-Cas9 in knocking out TRAC is shown in FIGS. 2A-2B. The results showed that N-oligo DNA can effectively increase the efficiency of CRISPR-Cas9 in knocking out the TRAC gene in both T cells and CAR-T cells.

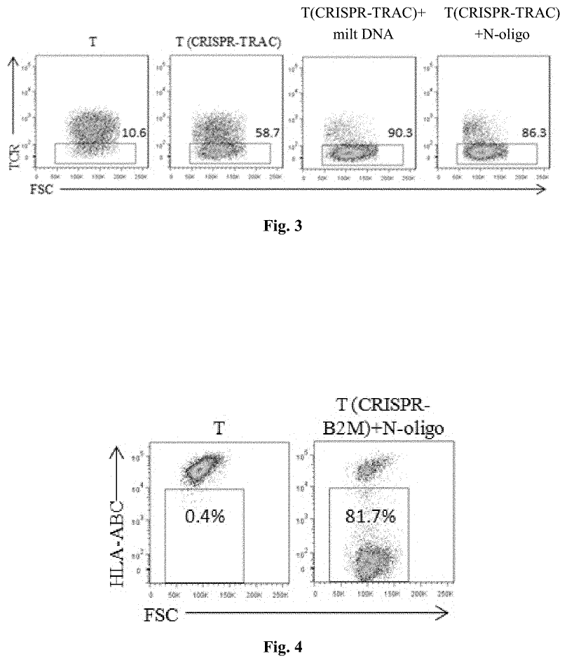

On the basis of the disclosure of Example 5 section (3), 100-200 nM milt DNA fragments were additionally added to the RNP complex, and the effect of the milt DNA fragments on the efficiency in knocking out TRAC is shown in FIG. 3. The results showed that the milt DNA fragments increased the efficiency of knocking out the TRAC gene, and the efficiency was higher than that of N-oligo.

### Test Example 4: Detection of Efficiency in Knocking Out B2M, PD1 in T Cells

Similarly, a number of crRNAs were designed, and after comparative experiment, the crRNA with the highest knockout efficiency and the lowest off-target rate was chosen for knocking out the B2M gene. B2M and/or PD1 genes were knocked out of T cells by using the RNP delivery system and N-oligo, based on the same method as that described in Example 5 (3).

For B2M protein, the efficiency in knocking out the B2M gene was detected by using APC-HLA-ABC antibody (eBioscience), because the expression of B2M gene was closely related to the display of HLA-ABC on cell membrane. The results (shown in FIG. 4) showed that the efficiency in knocking out B2M gene was greater than 80%.

For the PD1 gene, the cells were electroporated with the mixture of RNP and N-oligo for 48 hours, and 1×10⁶ cells were taken, washed twice with PBS buffer, and the supernatant was completely aspirated. T7E1 experiment was performed according to the manual of GeneArt® Genomic Cleavage Detection Kit (Thermo Fisher), and the results (shown in FIG. 5) showed that the three crRNAs selected for PD1 were all able to effectively knock out the PD1 gene, and the knockout efficiencies were all greater than 80%.

### Test Example 5: Analysis of Gene Mutations Caused by CRISPR-Cas9

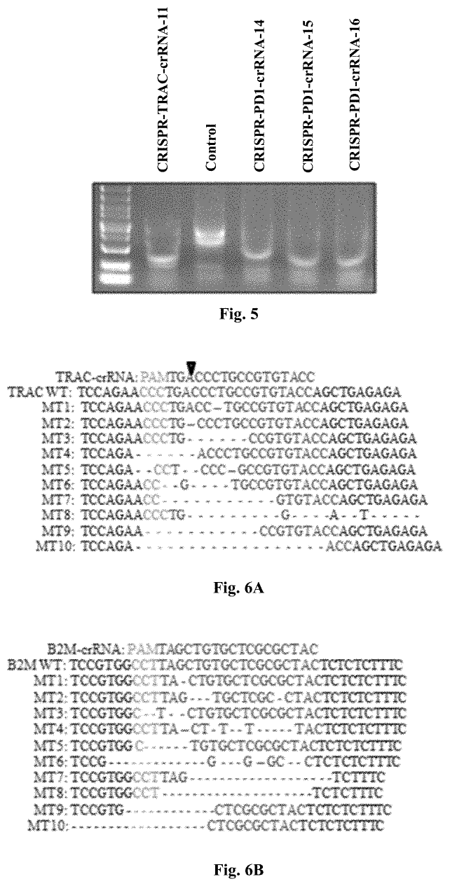

Primers were first designed near the target sites in TRAC, B2M and PD1 genes. After TRAC, B2M and PD1 were knocked out in T cells based on the CRISPR-Cas9 system, using RNP+N-oligo or milt DNA fragments. Genomic DNAs were extracted from 1×10⁶ of both normal T cells and T cells in which a gene has been knocked out, respectively. The obtained PCR product DNA fragment was ligated to a vector with T-blunt ends (pEASY-Blunt Simple Cloning Kit, Beijing TransGen Biotech Co., Ltd.), then the obtained vector was transformed into TOP10 competent cells which were plated onto the Amp-resistant solid plates. The next day, the obtained clones were sequenced, and at least 30 clones per plate were sequenced. The obtained sequencing results were aligned to the wild type sequence, and the results (as shown in FIG. 6A to FIG. 6B, the results for PD1 mutation are not shown) showed that CRISPR-Cas9 resulted in gene mutations in three genes at the genomic DNA sites corresponding to the crRNA, respectively.

### Test Example 6: Analysis of Off-Target

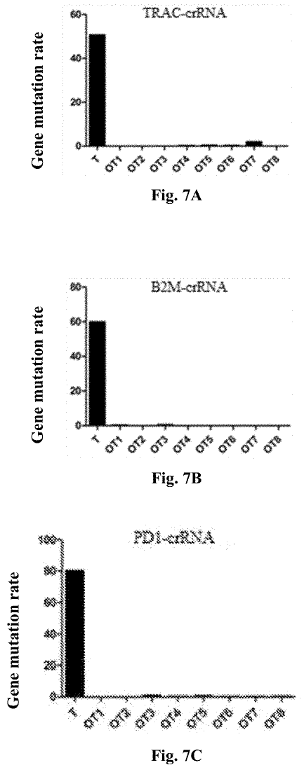

The possible off-target sites were predicted at http://crispr.mit.edu/, based on the designed crRNA. Eight or nine potential off-target sites (OT1-OT9) were selected for TRAC, B2M and PD1, respectively. Primers were designed for these potential off-target sites for PCR amplification and sequencing. The peak mapping sequencing results for off-target sites of the genomic DNA knocked out in cells and for controls (target gene TRAC, B2M or PD1) were aligned by TIDE on the website tide.nki.nl, and the results are shown in FIGS. 7A to 7C, indicating the off-target rate of the selected crRNA and the knockout method was extremely low.

### Test Example 7: Analysis of the Effect of TCR Knockout on Both Signaling Pathway and Killing Activity of the Cells

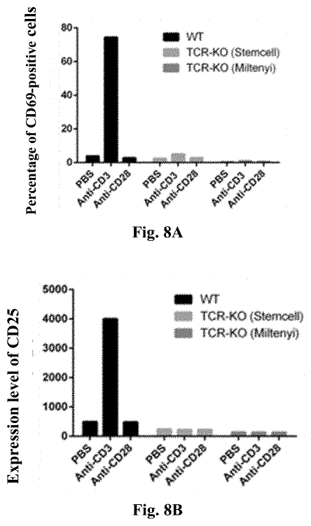

CD3 antibody solution (5 μg/ml) was prepared and coated on a 96-well plate. A volume of 100 μl was added to each well, and coated at 37° C. for two hours. The plated was taken out and washed twice with PBS. TCR-negative T cells and normal T cells were added respectively, and the cell density was 1×10⁶ cells/ml. After incubation at 37° C. for 24 hours, the plate was taken out and stained with antibodies specific for CD25 and CD69, and the results (as shown in FIG. 8A and FIG. 8B) showed that T cells in which the TRAC gene was knocked out cannot be induced to express CD25 and CD69 by CD3 antibody. Comparison of the killing effects of TCR-positive CAR-T and TCR-negative (such as knockout of TRAC) CAR-T cells on the CD19-positive tumor cell line K562-CD19, showed that TCR knockout does not significantly impact the killing effect of CAR-T cells.

For clear understanding, the invention has been described in detail with the aid of the drawings and examples provided herein, however the description and examples should not be construed as limiting the scope of the invention. The disclosures of all patents and scientific literatures cited herein are expressly incorporated by reference in their entirety.

1. A method for knocking out one or more target gene(s) in T cells in vitro, comprising:
1) contacting sgRNAs targeting one or more target gene(s) in the T cells with a Cas9 protein, respectively, to form a protein-RNA complex comprising each of the sgRNAs and the Cas9;
2) mixing the protein-RNA complex with an oligo deoxyribonucleic acid or a milt DNA fragment to obtain a mixture; and
3) transforming the mixture into T cells,
wherein the sgRNAs direct the Cas9 protein to the corresponding target sequences of the target gene and to hybridize with the target sequence, thereby the target gene is cleaved, and the cleavage efficiency of the target gene is greater than 75%.

2. The method according to claim 1, wherein the target gene is one or more genes selected from the group consisting of TRAC, TRBC, B2M and PD1 genes, and each of the sgRNAs targets a coding sequence of the target gene or a regulatory sequence for the expression of the target gene.

3. The method according to claim 1, wherein each of the sgRNAs consists of, from 5′ to 3′, a crRNA targeting the target gene and having 17-20 nucleotides (nt) in length, linked to a tracrRNA corresponding to the Cas9 protein.

4. The method according to claim 1, wherein the oligo deoxyribonucleic acid is a double-stranded DNA with a length of 100-250 base pairs (bp) or a single-stranded DNA with a length of 100-250 nt.

5. The method according to claim 3, wherein:
the crRNA targets the TRAC gene and comprises any one or more sequences selected from the group consisting of SEQ ID NOs: 1-12,
the crRNA targets the B2M gene and comprises the sequence of SEQ ID NO: 13, and/or
the crRNA targets the PD1 gene and comprises one or more sequence selected from the group consisting of SEQ ID NOs: 14-16.

6. The method according to claim 1, wherein the Cas9 protein is a Cas9 protein derived from Streptococcus pyogenes comprising the amino acid sequence of SEQ ID NO: 18.

7. The method according to claim 3, wherein the tracrRNA corresponding to the Cas9 protein comprises the nucleotide sequence of SEQ ID NO: 17.

8. The method according to claim 1, wherein the T cells are selected from the group consisting of helper T cells, cytotoxic T cells, memory T cells, regulatory T cells, natural killer T cells, γδT cells, CAR-T cells and TCR-T cells.

9. T cells with one or more target gene(s) knocked out, wherein in the T cells are obtained by the method according to claim 1.

10. A crRNA for use in knocking out a target gene, wherein the crRNA comprises one or more sequences selected from the group consisting of SEQ ID NOs: 1-16.

11. The crRNA according to claim 10, wherein the target gene is one or more genes selected from the group consisting of TRAC, TRBC, B2M and PD1 genes.

12. The crRNA according to claim 11, wherein the target gene is the TRAC gene, and the crRNA comprises one or more sequences selected from the group consisting of SEQ ID NOs: 1-12.

13. The crRNA according to claim 11, wherein the target gene is the B2M gene, and the crRNA comprises the sequence of SEQ ID NO: 13.

14. The crRNA according to claim 11, wherein the target gene is the PD1 gene, and the crRNA comprises one or more sequences selected from the group consisting of SEQ ID NOs: 14-16.

15. A sgRNA for use in knocking out a target gene, wherein the sgRNA consists of a crRNA linked to a tracrRNA corresponding to a Cas9 protein, and wherein the crRNA comprises one or more sequences selected from the group consisting of SEQ ID NOs: 1-16.

16. The sgRNA according to claim 15, wherein the target gene is one or more gene selected from the group consisting of TRAC, TRBC, B2M and PD1 genes.

17. The sgRNA according to claim 16, wherein the target gene is the TRAC gene, and the crRNA comprises one or more sequences selected from the group consisting of SEQ ID NOs: 1-12.

18. The sgRNA according to claim 16, wherein the target gene is the B2M gene, and the crRNA comprises the sequence of SEQ ID NO: 13.

19. The sgRNA according to claim 16, wherein the target gene is the PD1 gene, and the crRNA comprises one or more sequences selected from the group consisting of SEQ ID NOs: 14-16.

20. The sgRNA according to claim 15, wherein the Cas9 protein is a Cas9 protein derived from Streptococcus pyogenes comprising the amino acid sequence of SEQ ID NO: 18.

21. The sgRNA according to claim 15, wherein the tracrRNA corresponding to the Cas9 protein comprises the nucleotide sequence of SEQ ID NO: 17.

22. A kit for use in knocking out a target gene comprising:
a) a crRNA according to claim 10;
b) a Cas9 protein; and
c) an oligo deoxyribonucleic acid or a milt DNA fragment.

23. The kit according to claim 22, wherein the oligo deoxyribonucleic acid is a double-stranded DNA with a length of 100-250 bp or a single-stranded DNA with a length of 100-250 nt.

24. The kit according to claim 22, wherein the Cas9 protein is a Cas9 protein derived from Streptococcus pyogenes comprising the amino acid sequence of SEQ ID NO: 18.

25. (canceled)

26. (canceled)

27. A method of treating a tumor, comprising administering to a subject in need thereof the T cells with the target gene knockout of claim 9.

28. A method of preventing or treating an infectious disease caused by a virus or bacterium, comprising administering to a subject in need thereof the T cells with the target gene knockout of claim 9.


SEQUENCE LISTING (19 sequences)

SEQ ID NO: 1 - RNA, 18 nt, artificial sequence
  crRNA-1
caaaugugucacaaagua

SEQ ID NO: 2 - RNA, 18 nt, artificial sequence
  crRNA-2
aaaacugugcuagacaug

SEQ ID NO: 3 - RNA, 18 nt, artificial sequence
  crRNA-3
ucaagagcaacagugcug

SEQ ID NO: 4 - RNA, 18 nt, artificial sequence
  crRNA-4
caccuucuuccccagccc

SEQ ID NO: 5 - RNA, 18 nt, artificial sequence
  crRNA-5
gaauaaugcuguuguuga

SEQ ID NO: 6 - RNA, 18 nt, artificial sequence
  crRNA-6
gauuuagagucucucagc

SEQ ID NO: 7 - RNA, 18 nt, artificial sequence
  crRNA-7
acggcagggucaggguuc

SEQ ID NO: 8 - RNA, 18 nt, artificial sequence
  crRNA-8
guuccugugaugucaagc

SEQ ID NO: 9 - RNA, 18 nt, artificial sequence
  crRNA-9
ucaaaaccugucagugau

SEQ ID NO: 10 - RNA, 18 nt, artificial sequence
  crRNA-10
gaauccuccuccugaaag

SEQ ID NO: 11 - RNA, 17 nt, artificial sequence
  crRNA-11
gguacacggcaggguca

SEQ ID NO: 12 - RNA, 20 nt, artificial sequence
  crRNA-12
gagaaucaaaaucggugaau

SEQ ID NO: 13 - RNA, 18 nt, artificial sequence
  crRNA-13
guagcgcgagcacagcua

SEQ ID NO: 14 - RNA, 20 nt, artificial sequence
  crRNA-14
cgacuggccagggcgccugu

SEQ ID NO: 15 - RNA, 18 nt, artificial sequence
  crRNA-15
gugcuacaacugggcugg

SEQ ID NO: 16 - RNA, 20 nt, artificial sequence
  crRNA-16
ggcgcccuggccagucgucu

SEQ ID NO: 17 - RNA, 80 nt, artificial sequence
  tracrRNA
guuuuagagcuagaaauagcaaguuaaaauaaggcuaguccguuaucaacuugaaaaaguggcaccgagucggugcuuuu

SEQ ID NO: 18 - protein, 1378 aa, artificial sequence
  Amino acid sequence of the Cas9(including NLS) protein
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVMDK

SEQ ID NO: 19 - DNA, 127 bp, artificial sequence
  random N-oligo
tcatgtggtcggggtagcggctgaagcactgcacgccgtacgtcagggtggtcacgagggtgggccagggcacgggcagcttgccggtggtgcagatgaacttcagggtcagcttgccgtaggtggc